(19) United States
(12) Patent Application Publication
(10) Pub. No.: US 2017/0252009 A1
(43) Pub. Date: Sep. 7, 2017

(54) ULTRASONIC DIAGNOSTIC APPARATUS AND PROGRAM FOR CONTROLLING THE SAME

(71) Applicant: General Electric Company

(72) Inventors: Sotaro Kawae, Tokyo (JP); Hiroshi Hashimoto, Tokyo (JP)

(21) Appl. No.: 15/506,510

(22) PCT Filed: Aug. 26, 2015
(86) PCT No.: PCT/US15/46879

§ 371 Date: Feb. 24, 2017

(30) Foreign Application Priority Data
Aug. 27, 2014 (JP) 2014-173056

(51) Int. Cl. A61B 8/00; A61B 8/14; G01S 7/52; A61B 8/08

(52) U.S. Cl. CPC A61B 8/461; A61B 8/485; A61B 8/14; A61B 8/5207; G01S 7/52042; G01S 7/52073

(57) ABSTRACT

An ultrasonic diagnostic apparatus is provided for allowing a displacement between a direction of an acoustic line of ultrasound and a direction of movement of biological tissue to be recognized. The apparatus includes a strain calculating section for calculating a strain in biological tissue based on two temporally different echo signals in an identical acoustic line acquired by an ultrasonic probe; an elasticity image data generating section for generating data for an elasticity image according to the strain calculated by the strain calculating section; a movement detecting section for detecting movement of the biological tissue in a B-mode image; an angle calculating section for calculating an angle between a direction of an acoustic line of ultrasound transmitted/received by the ultrasonic probe and a direction of movement of the biological tissue detected by the movement detecting section; and an image display processing section for displaying an indicator indicating the angle.

### TECHNICAL FIELD

Embodiments of the present invention relate to an ultrasonic diagnostic apparatus and a program for controlling the same with which an elasticity image representing hardness or softness of biological tissue in a subject is displayed.

### BACKGROUND

An ultrasonic diagnostic apparatus for displaying an elasticity image representing hardness or softness of biological tissue in a subject in combination with a B-mode image is disclosed in Patent Document 1 (Japanese Patent Application KOKAI No. 2007-282932), for example. The elasticity image is produced as follows, for example. First, ultrasound is transmitted to the subject, and a physical quantity related to elasticity of a subject is calculated based on resulting echo signals. Based on the calculated physical quantity, an elasticity image composed of colors corresponding to the elasticity is produced for display.

The physical quantity related to elasticity is strain, for example. Patent Document 2 (Japanese Patent Application KOKAI No. 2008-126079) discloses a technique of estimating a strain by acquiring two temporally different echo signals in an identical acoustic line by an ultrasonic probe, and comparing waveforms of the acquired echo signals to estimate a strain in a direction of the acoustic line of ultrasound based on a degree of distortion of the waveforms associated with compression and relaxation of the biological tissue between the two echo signals.

### BRIEF DESCRIPTION

In recent years, there has been a need for evaluation of hepatic diseases by an ultrasonic diagnostic apparatus capable of displaying elasticity images. The present disclosure relates to the production of an elasticity image using a strain of a liver brought about by pulsation of a heart and/or blood vessels.

Such a technique as that disclosed in Patent Document 2 of calculating a strain of biological tissue by a degree of distortion of waveforms of echo signals associated with compression and relaxation of the biological tissue calculates the strain in a direction of an acoustic line of ultrasound. Therefore, in calculating a strain of biological tissue by a degree of distortion of waveforms of echo signals associated with compression and relaxation of the biological tissue, an accurate strain probably cannot be calculated in case that the direction of an acoustic line of ultrasound does not match a direction in which deformation is brought about in biological tissue by pulsation of a heart and/or blood vessels.

Embodiments of the invention made for solving the problem described above include an ultrasonic diagnostic apparatus comprising an ultrasonic probe for conducting transmission/reception of ultrasound to/from biological tissue; a strain calculating section for calculating a strain in several portions in said biological tissue based on two temporally different echo signals in an identical acoustic line acquired by said ultrasonic probe, said section calculating said strain in a direction of said acoustic line of ultrasound; an elasticity image data generating section for generating data for an elasticity image according to the strain calculated by said strain calculating section; a movement detecting section for detecting movement of said biological tissue in an ultrasonic image based on ultrasonic image data generated based on echo signals resulting from transmission/reception of ultrasound to/from said biological tissue; an angle calculating section for calculating an angle between a direction of an acoustic line of ultrasound transmitted/received by said ultrasonic probe and a direction of movement of said biological tissue detected by said movement detecting section; and a notifying section for notifying information based on the angle calculated by said angle calculating section.

According to an embodiment of the invention, information based on an angle between a direction of an acoustic line of ultrasound transmitted/received by the ultrasonic probe and a direction of movement of the biological tissue detected by the movement detecting section is notified, an operator can recognize a displacement between the direction of the acoustic line of ultrasound and direction of movement of the biological tissue.

### BRIEF DESCRIPTION OF THE DRAWINGS

FIG. 1 is a block diagram showing an exemplary configuration of an embodiment of an ultrasonic diagnostic apparatus in accordance with an embodiment of the present invention.

FIG. 2 is a block diagram showing a configuration of an echo data processing section in the ultrasonic diagnostic apparatus shown in FIG. 1.

FIG. 3 is a block diagram showing a configuration of a display processing section in the ultrasonic diagnostic apparatus shown in FIG. 1.

FIG. 4 is a diagram showing a display section displaying a combined ultrasonic image having a B-mode image and an elasticity image combined together.

FIG. 5 is a diagram showing the display section displaying an indicator along with the combined ultrasonic image.

FIG. 6 is a flow chart explaining display of the indicator in the first embodiment.

FIG. 7 is a diagram showing a plurality of sub-regions defined in a region of interest.

FIG. 8 is a diagram showing motion vectors detected respectively for the plurality of sub-regions.

FIG. 9 is an enlarged view of the indicator.

FIG. 10 is a diagram explaining a range in which a solid line pivotally moves in the indicator.

FIG. 11 is a diagram showing the display section displaying characters representing an angle in a variation of the first embodiment.

FIG. 12 is a block diagram showing an exemplary configuration of an embodiment of the ultrasonic diagnostic apparatus having a speaker.

FIG. 13 is a flow chart explaining display of elasticity images in a plurality of sub-regions in a second embodiment.

FIG. 14 is a diagram showing the display section displaying combined color elasticity images respectively in the plurality of sub-regions.

FIG. 15 is a diagram showing the display section having some of the plurality of sub-regions displaying no combined color elasticity images therein in a variation of the second embodiment.

FIG. 16 is a block diagram showing a configuration of a display processing section in an ultrasonic diagnostic apparatus in a third embodiment.

FIG. 17 is a flow chart explaining an operation in the third embodiment.

FIG. 18 is a diagram showing the display section displaying combined color movement-amount images produced based on movement-amount image data.

FIG. 19 is a diagram showing the display section having a region of interest defined.

FIG. 20 is a diagram showing the display section displaying a combined color elasticity image in the third embodiment.

### DETAILED DESCRIPTION

Now embodiments of the present invention will be described with reference to the accompanying drawings.

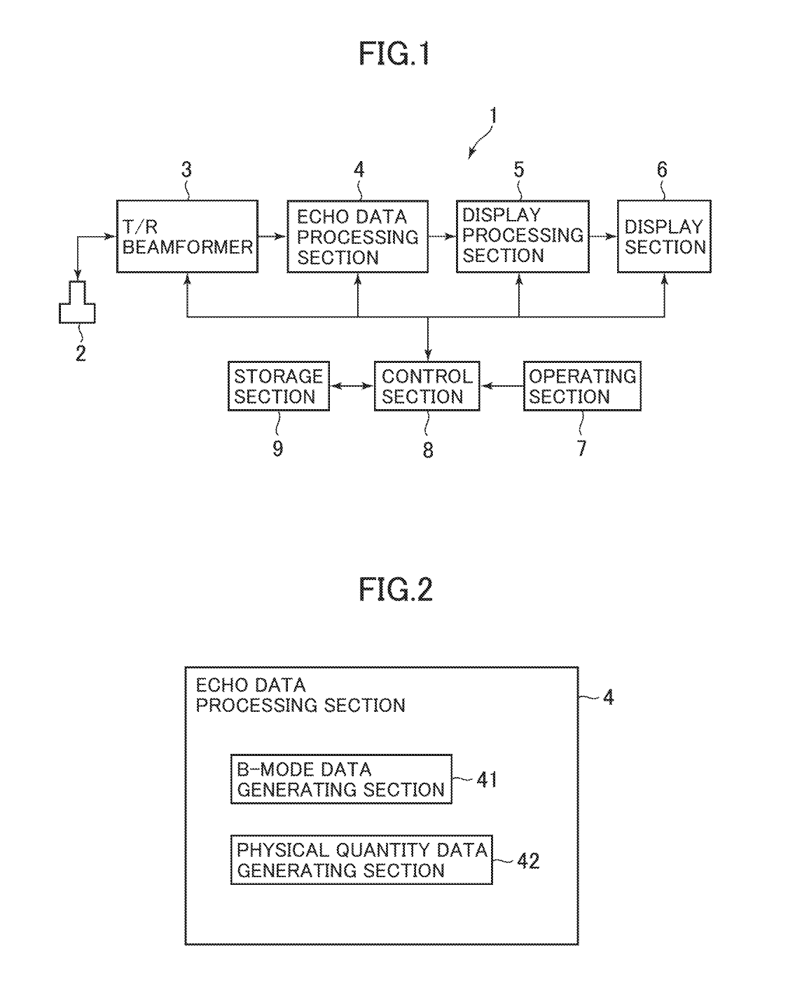

To begin with, a first embodiment will be described. An ultrasonic diagnostic apparatus 1 shown in FIG. 1 comprises an ultrasonic probe 2, a transmission/reception (T/R) beamformer 3, an echo data processing section 4, a display processing section 5, a display section 6, an operating section 7, a control section 8, and a storage section 9. The ultrasonic diagnostic apparatus 1 has a configuration as a computer.

The ultrasonic probe 2 is configured to comprise a plurality of ultrasonic vibrators (not shown) arranged in an array, and ultrasound is transmitted to a subject and echo signals thereof are received by the ultrasonic vibrators. The ultrasonic probe 2 represents an exemplary embodiment of the ultrasonic probe in the present invention.

The T/R beamformer 3 supplies an electric signal to the ultrasonic probe 2 for transmitting ultrasound from the ultrasonic probe 2 with specified scan conditions based on a control signal from the control section 8. The T/R beamformer 3 also applies signal processing such as A/D conversion and phased addition processing to echo signals received by the ultrasonic probe 2, and outputs echo data after the signal processing to the echo data processing section 4.

The echo data processing section 4 comprises a B-mode data generating section 41 and a physical quantity data generating section 42, as shown in FIG. 2. The B-mode data generating section 41 applies B-mode processing such as logarithmic compression processing and envelope detection processing to the echo data output from the T/R beamformer 3, and generates B-mode data. The B-mode data may be stored in the storage section 9.

The physical quantity data generating section 42 calculates a physical quantity related to elasticity in several portions in the subject, and generates physical quantity data based on the echo data output from the T/R beamformer 3 (physical quantity calculating function). The physical quantity data generating section 42 defines a correlation window for temporally different echo data in an identical acoustic line in one scan plane, applies correlation calculation between correlation windows to calculate a physical quantity related to elasticity on a pixel-by-pixel basis, and generates physical quantity data in one frame, as described in Japanese Patent Application KOKAI No. 2008-126079, for example. Therefore, echo data in two frames yields physical quantity data in one frame, and an elasticity image is produced as will be discussed later. The physical quantity data may be stored in the storage section.

The physical quantity data generating section 42 calculates a strain of biological tissue by a degree of distortion of waveforms of echo signals associated with compression and relaxation of the biological tissue by the correlation calculation between correlation windows. Therefore, the physical quantity related to elasticity is a strain here, and strain data is obtained as the physical quantity data.

In the present embodiment, a strain due to deformation of a liver by pulsation of a heart and/or blood vessels is calculated, as will be discussed later. The strain obtained here by the physical quantity data generating section 42 is a strain in a direction of an acoustic line of ultrasound. In case that a direction of deformation (direction of movement) of the liver is different from the direction of an acoustic line of ultrasound, a strain of a component in the acoustic line direction within an actual strain is calculated by the physical quantity data generating section 42. Therefore, as an angle between the direction of deformation of the liver and direction of the acoustic line of ultrasound increases, a difference between the strain calculated by the physical quantity data generating section 42 and actual strain becomes greater.

The physical quantity data generating section 42 represents an exemplary embodiment of the strain calculating section in the present invention. The physical quantity calculating function represents an exemplary embodiment of the strain calculating function in the present invention.

When a region of interest R is defined in a B-mode image as will be discussed later, the physical quantity data generating section 42 may perform the calculation of a strain for the region of interest R.

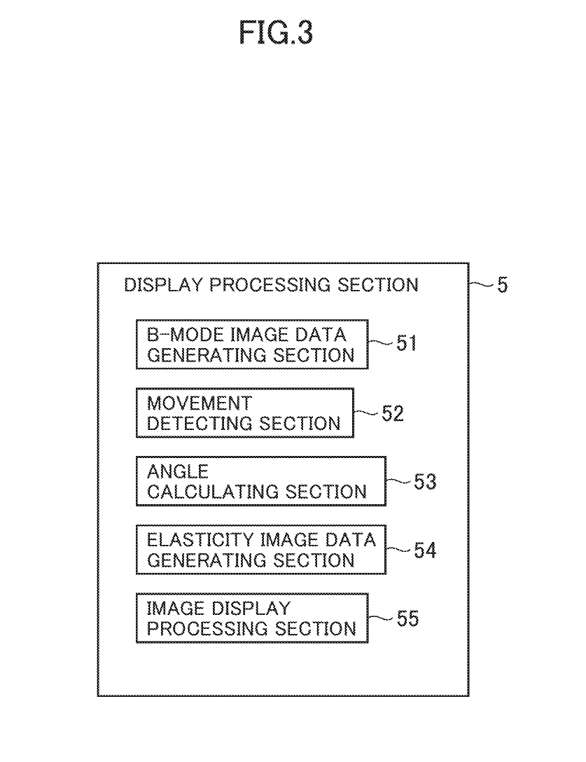

The display processing section 5 comprises a B-mode image data generating section 51, a movement detecting section 52, an angle calculating section 53, an elasticity image data generating section 54, and an image display processing section 55, as shown in FIG. 3. The B-mode image data generating section 51 applies scan conversion to B-mode data by a scan converter to convert the data into B-mode image data having information representing brightness according to the intensity of echo signals. The B-mode image data has information representing brightness at 256 levels, for example.

The movement detecting section 52 detects movement of biological tissue in a B-mode image based on the B-mode image data (movement detecting function). Details thereof will be discussed later. The movement detecting section 52 represents an exemplary embodiment of the movement detecting section in the present invention. The movement detecting function represents an exemplary embodiment of the movement detecting function in the present invention.

The angle calculating section 53 calculates an angle between the direction of an acoustic line of ultrasound transmitted/received by the ultrasonic probe 2 and the direction of movement of the biological tissue detected by the movement detecting section 52 (angle calculating function). The angle calculating section 53 represents an exemplary embodiment of the angle calculating section in the present invention. The angle calculating function represents an exemplary embodiment of the angle calculating function in the present invention.

The elasticity image data generating section 54 transforms the physical quantity data into information representing colors, and applies scan conversion by the scan converter to generate elasticity image data having information representing colors according to the strain (elasticity image data generating function). The elasticity image data generating section 54 also gives multiple gradations to the physical quantity data, and generates elasticity image data comprised of information representing colors assigned to the gradations. The elasticity image data generating section 54 represents an exemplary embodiment of the elasticity image data generating section in the present invention. The elasticity image data generating function represents an exemplary embodiment of the elasticity image data generating function in the present invention.

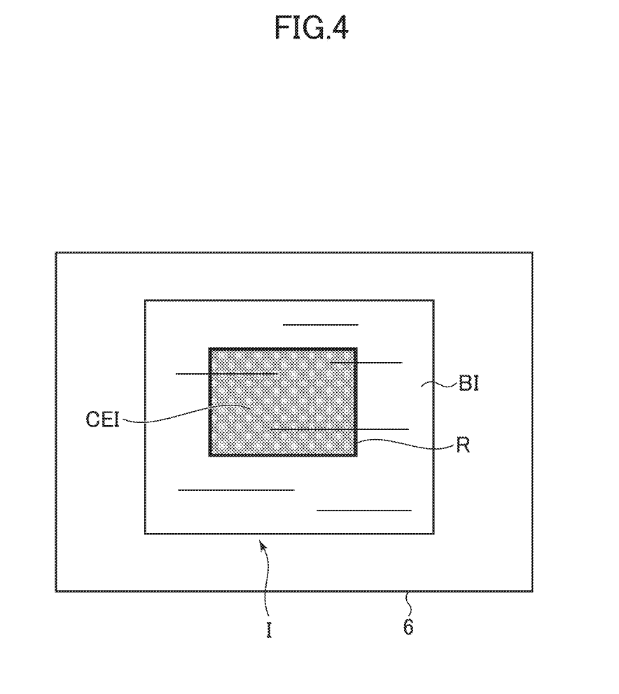

The image display processing section 55 combines the B-mode image data with the elasticity image data in a specified proportion in the region of interest R to generate image data for an image to be displayed in the display section 6. Based on the image data, the image display processing section 55 then displays an image I in the region of interest R having the combined color elasticity image CEI obtained by combining the B-mode image data with the elasticity image data in the display section 6 (image display control function), as shown in FIG. 4.

The image I has the combined color elasticity image CEI displayed in the region of interest R defined on the B-mode image BI. The combined color elasticity image CEI is a color image through which the B-mode image in the background is visible. The combined color elasticity image CEI has a degree of transparency according to the proportion of combination of the B-mode image data and elasticity image data. The combined color elasticity image CEI is an elasticity image having colors according to the strain and representing elasticity of the biological tissue.

The B-mode image data and elasticity image data may be stored in the storage section 9. The image data of a combination of the B-mode image data and elasticity image data may also be stored in the storage section 10.

The image display processing section 55 displays information based on the angle calculated by the angle calculating section 53 in the display section 6. Details thereof will be discussed later. The image display processing section 55 represents an exemplary embodiment of the notifying section in the present invention.

The display section 7 is an LCD (Liquid Crystal Display) or an organic EL (Electro-Luminescence) display, for example.

The operating section 7 is configured to comprise a keyboard for allowing an operator to input a command and/or information, a pointing device, and the like (not shown).

The control section 8 is a processor such as a CPU (Central Processing Unit). The control section 8 loads thereon a program stored in the storage section 9 and controls several sections in the ultrasonic diagnostic apparatus 1. For example, the control section 8 loads thereon a program stored in the storage section 9 and executes functions of the T/R beamformer 3, echo data processing section 4, and display processing section 5 by the loaded program.

The control section 8 may execute all of the functions of the T/R beamformer 3, all of the functions of the echo data processing section 4, and all of the functions of the display processing section 5 by the program, or execute only some of the functions by the program. In case that the control section 8 executes only some of the functions, the remaining functions may be executed by hardware such as circuitry.

It should be noted that the functions of the T/R beamformer 3, echo data processing section 4, and display processing section 5 may be implemented by hardware such as circuitry.

The storage section 9 is an HDD (Hard Disk Drive), and/or a semiconductor memory such as a RAM (Random Access Memory) and/or a ROM (Read-Only Memory). The ultrasonic diagnostic apparatus 1 may comprise all of the HDD, RAM, and ROM for the storage section 9. The storage section 9 may also be a portable storage medium such as a CD (Compact Disk) or a DVD (Digital Versatile Disk).

The program executed by the control section 8 is stored in a non-transitory storage medium such as the HDD or ROM described above. The program may also be stored in a non-transitory portable storage medium such as the CD or DVD described above.

Now an operation of the ultrasonic diagnostic apparatus 1 in the present embodiment will be described below. The T/R beamformer 3 causes the ultrasonic probe 2 to transmit ultrasound to biological tissue in a subject. In the present embodiment, the ultrasonic probe 2 transmits ultrasound to a liver in a subject.

The T/R beamformer 3 may cause ultrasound for generating B-mode image data and that for generating elasticity image data to be alternately transmitted. Echo signals of the ultrasound transmitted from the ultrasonic probe 2 are received by the ultrasonic probe 2.

The liver repetitively deforms due to pulsation of the heart and/or blood vessels. An elasticity image is produced based on echo signals obtained from the repetitively deforming liver by capturing the deformation as strain. In particular, once echo signals have been acquired, the B-mode data generating section 41 generates B-mode data, and the physical quantity data generating section 42 calculates a strain to generate physical quantity data. Moreover, the B-mode image data generating section 51 generates B-mode image data based on the B-mode data and the elasticity image data generating section 54 generates elasticity image data based on the strain data. The image display processing section 55 then displays an image I having a combined color elasticity image CEI obtained by combining the B-mode image data with the elasticity image data in the display section 6, as shown in FIG. 4 described above. The image I is a real-time image here.

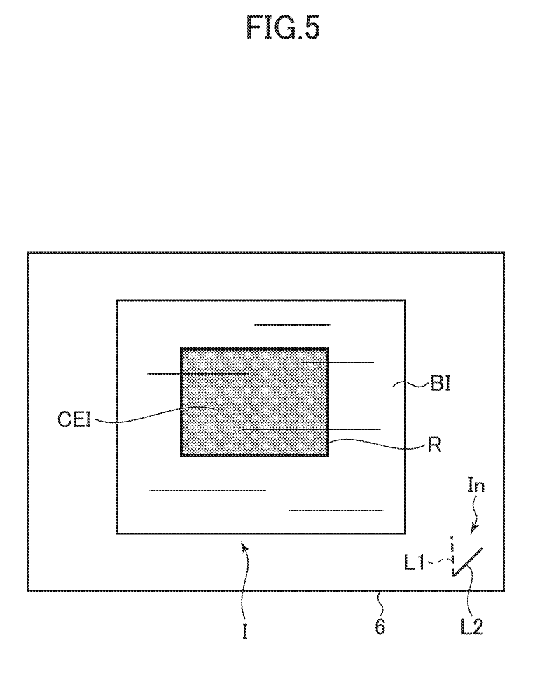

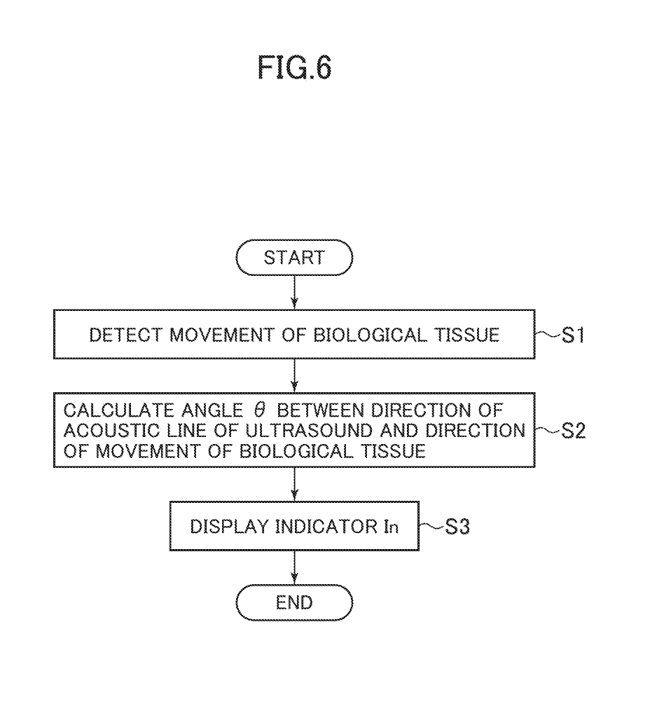

The image display processing section 55 also displays an indicator In along with the image I in the display section 6, as shown in FIG. 5. The indicator In is comprised of a dashed line L1 and a solid line L2. Display of the indicator In will now be described with reference to the flow chart in FIG. 6.

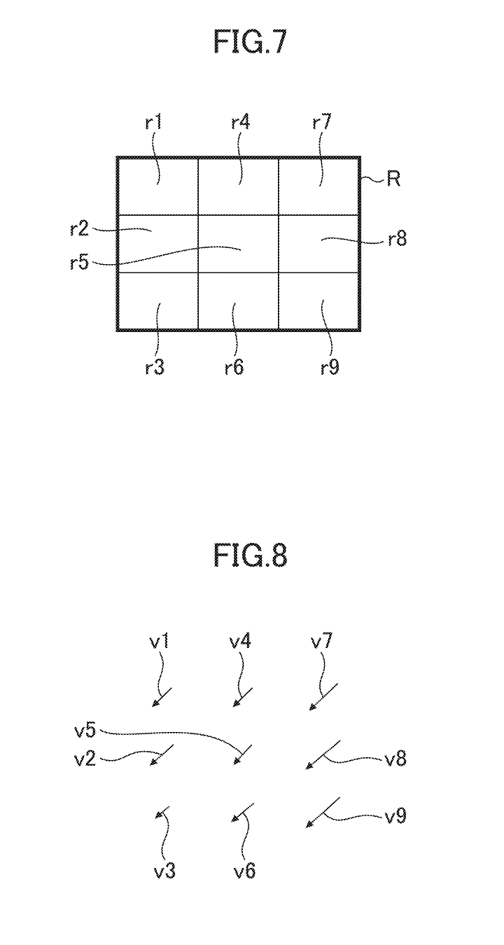

First, at Step S1, the movement detecting section 52 detects movement of biological tissue in the B-mode image BI. The movement detecting section 52 detects the movement of the biological tissue in the region of interest R. This will be particularly described. For example, the movement detecting section 52 first detects movement of the biological tissue in the B-mode image in each of a plurality of sub-regions r1-r9 defined in the region of interest R, as shown in FIG. 7. The movement detecting section 52 determines, in the B-mode image data in one of two temporally different frames for an identical cross section, to which portion each of the plurality of sub-regions r1-r9 has moved in the other of the frames by a known technique such as one using a degree of image similarity according to correlation calculation.

While the region of interest R is divided into nine sub-regions r1-r9 in FIG. 7, the number of sub-regions is not limited thereto.

The movement detecting section 52 thus detects movement for each of the plurality of sub-regions r1-r9 to thereby provide motion vectors v1-v9 respectively for the plurality of sub-regions r1-r9, as shown in FIG. 8. The movement detecting section 52 calculates an average vector Vav (not shown) of the motion vectors v1 v9. By the calculation of the average vector Vav, movement of the biological tissue in the region of interest R is detected.

Next, at Step S2, the angle calculating section 53 calculates an angle θ between the direction of the acoustic line of ultrasound and direction of movement of the biological tissue in the region of interest R detected at the movement detecting section 52. The direction of movement of the biological tissue is a direction of the average vector Vav calculated at Step S1 described above.

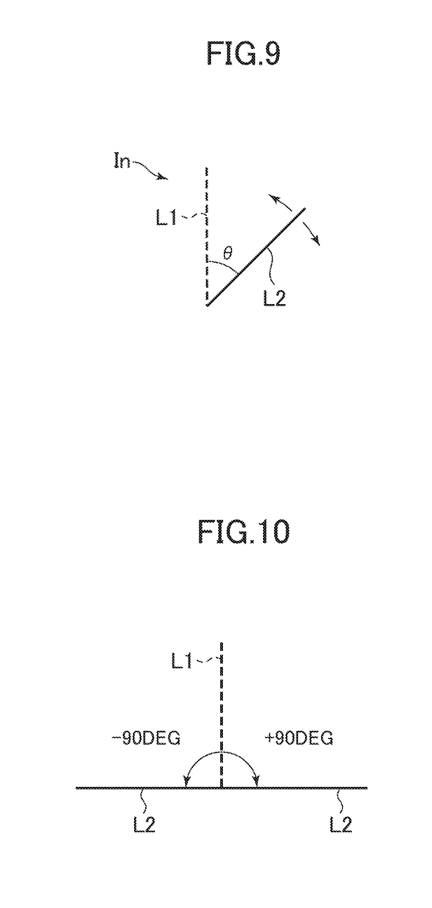

Next, at Step S3, the image display processing section 55 displays the indicator In in the display section 6 based on the angle θ calculated at Step S2 described above. In the indicator In, the dashed line L1 indicates a direction of an acoustic line of ultrasound and the solid line L2 indicates a direction of the average vector Vav (direction of movement of the biological tissue). As shown in FIG. 9, an angle formed by the dashed line L1 and solid line L2 is the angle θ. The indicator In is the information based on the angle in an embodiment of the present invention, information indicating an angle between the direction of the acoustic line of ultrasound and direction of movement of the biological tissue, and also information indicating a degree of match between the direction of the acoustic line of ultrasound and direction of movement of the biological tissue.

By the indicator In thus displayed, the operator can recognize a displacement between the direction of the acoustic line of ultrasound and direction of movement of the biological tissue. Therefore, the operator can adjust the angle or the like of the ultrasonic probe 2 so that the dashed line L1 matches the solid line L2 to thereby match the direction of the acoustic line of ultrasound with the direction of movement of the biological tissue. Therefore, the indicator In may be considered as information for the operator to recognize in which direction and at which angle to move the ultrasonic probe so that the direction of the acoustic line of ultrasound matches the direction of movement of the biological tissue.

More particularly, the processing at Steps S1-S3 described above is repetitively performed and display of the indicator In is updated. Therefore, once the operator has adjusted the angle or the like of the ultrasonic probe 2 to change the angle θ, the solid line L2 pivotally moves around an intersection thereof with the dashed line L1, as shown in FIG. 9. The operator can then adjust the angle or the like of the ultrasonic probe 2 while viewing the indicator In until the direction of the acoustic line of ultrasound matches the direction of movement of the biological tissue. Once the direction of the acoustic line of ultrasound has matched the direction of movement of the biological tissue, a combined color elasticity image CEI may be displayed, in which elasticity of the biological tissue is more accurately reflected.

Since the dashed line L1 is a direction of an acoustic line, it is displayed in the display section 6 at a vertically fixed position. Representing the position of the dashed line L1 displayed in such a direction as zero degree, the solid line L2 is displayed at a position up to 90 degrees clockwise and down to 90 degrees counterclockwise with respect to the dashed line L1, as shown in FIG. 10. The clockwise direction is positive while the counterclockwise direction is negative. Therefore, the angle θ is −90≦θ≦+90.

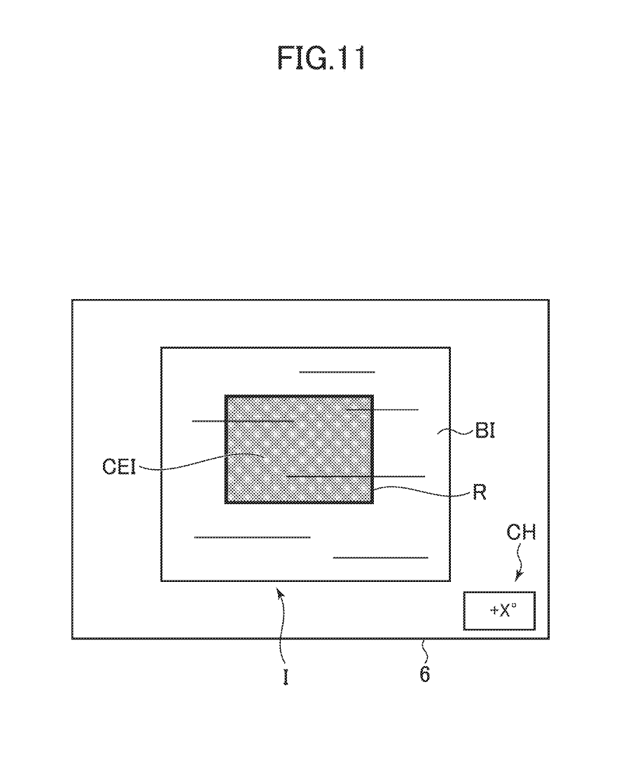

Next, a variation of the first embodiment will be described. The image display processing section 55 may display characters representing the angle θ, in place of the indicator In, in the display section 6. For example, the image display processing section 55 displays characters CH “+X^{o}” as characters indicating the angle θ (θ=X^{o}), as shown in FIG. 11.

The characters CH represent an exemplary embodiment of the information indicating an angle between the direction of the acoustic line of ultrasound and direction of movement of the biological tissue in the present invention, and also an exemplary embodiment of the information indicating a degree of match between the direction of the acoustic line of ultrasound and direction of movement of the biological tissue. The characters CH moreover represent an exemplary embodiment of the information for allowing an operator to understand in which direction and at which angle to move the ultrasonic probe so that the direction of the acoustic line of ultrasound matches the direction of movement of biological tissue the present invention.

The image display processing section 55 may display in which direction and at which angle to move the ultrasonic probe 2 in the display section 6 by characters, in place of the indicator In. The direction and angle in/at which the ultrasonic probe 2 is to be moved are those in/at which the ultrasonic probe 2 is to be moved so that the direction of the acoustic line of ultrasound matches the direction of movement of the biological tissue.

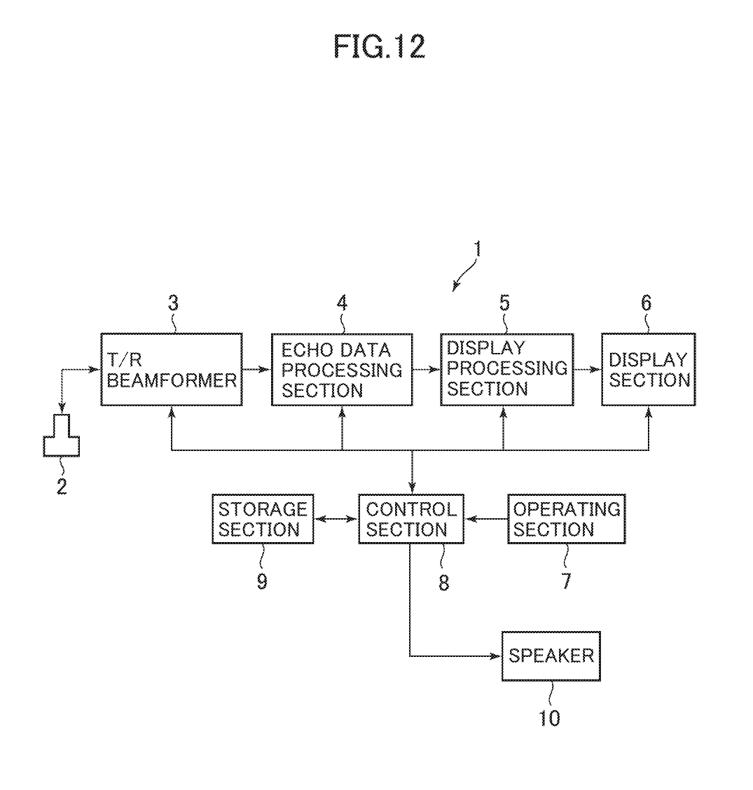

Moreover, the angle θ or the direction and angle in/at which the ultrasonic probe 2 is to be moved may be audibly notified. In this case, the control section 8 in the ultrasonic diagnostic apparatus 1 outputs voice from a speaker 10, as shown in FIG. 12. At that time, the control section 8 represents an exemplary embodiment of the notifying section in the present invention.

Next, a second embodiment will be described. It should be noted that identical parts to those in the first embodiment will be omitted in the description.

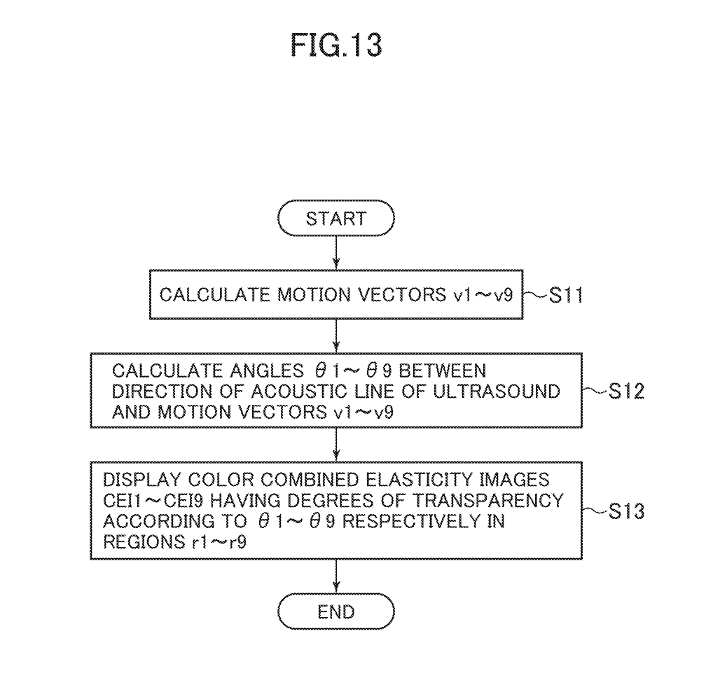

In the present embodiment, combined color elasticity images CEI1-CEI9 having respective degrees of transparency according to the angles θ1-θ9 between the direction of the acoustic line of ultrasound and directions of the vectors v1-v9 are displayed respectively in the plurality of sub-regions r1-r9. Now description will be made with reference to the flow chart in FIG. 13.

First, at Step S11, the movement detecting section 52 obtains motion vectors v1-v9 respectively for the plurality of sub-regions r1-r9, as in Step S1 described earlier. It should be noted that the movement detecting section 52 does not need to calculate the average vector Vav in the present embodiment.

Next, at Step S12, the angle calculating section 53 calculates an angle θ1 between the direction of the acoustic line of ultrasound and motion vector v1, an angle θ2 between the direction of the acoustic line of ultrasound and motion vector v2, an angle θ3 between the direction of the acoustic line of ultrasound and motion vector v3, an angle θ4 between the direction of the acoustic line of ultrasound and motion vector v4, an angle θ5 between the direction of the acoustic line of ultrasound and motion vector v5, an angle θ6 between the direction of the acoustic line of ultrasound and motion vector v6, an angle θ7 between the direction of the acoustic line of ultrasound and motion vector v7, an angle θ8 between the direction of the acoustic line of ultrasound and motion vector v8, and an angle θ9 between the direction of the acoustic line of ultrasound and motion vector v9. The angles θ1-θ9 are −90≦θ1-θ≦+90.

Next, at Step S13, the image display processing section 55 generates data of the combined color elasticity image CEI having respective degrees of transparency of the B-mode image BI according to the angles θ1-θ9 in the plurality of sub-regions r1-r9. Thus, data of combined color elasticity images CEI1-CEI9 are generated respectively for the plurality of sub-regions r1-r9.

For example, the elasticity image data generating section 54 increases the proportion of incorporation of the B-mode image data and decreases that of the elasticity image data for a greater absolute value of the angle θ1-θ9. Thus, the degree of transparency of the B-mode image is increased. On the other hand, the elasticity image data generating section 54 decreases the proportion of incorporation of the B-mode image data and increases that of the elasticity image data for a smaller absolute value of the angle θ1-θ9. Thus, the degree of transparency of the B-mode image is lowered.

Therefore, the proportion of incorporation of the B-mode image data is lowest for θ1-θ9 of zero degree and highest for an absolute value of θ1-θ9 of 90 degrees. On the other hand, the proportion of incorporation of the elasticity image data is highest for θ1-θ9 of zero degree and lowest for an absolute value of θ1θ9 of 90 degrees.

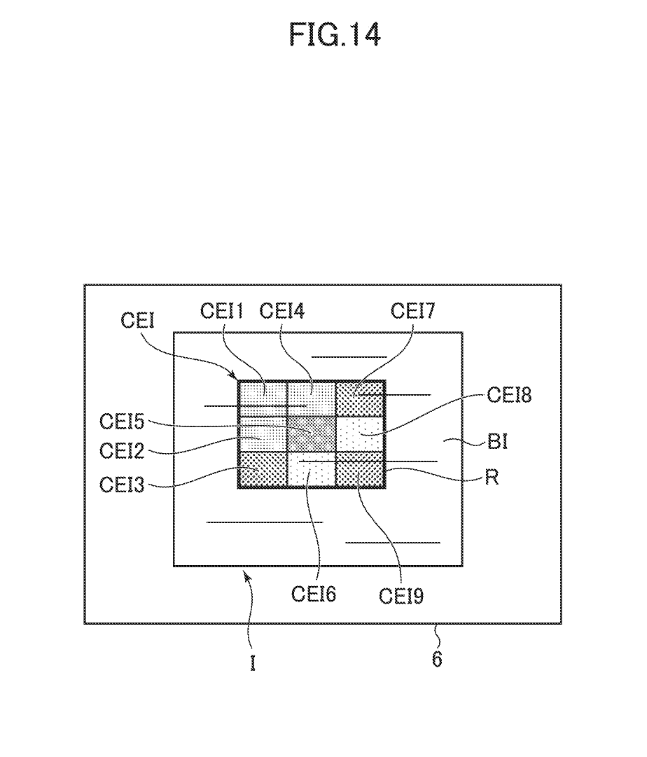

Once data for the combined color elasticity images CEI1-CEI9 having respective degrees of transparency of the B-mode image BI according to the angles θ1-θ9 have been produced, the image display processing section 55 displays the combined color elasticity images CEI1-CEI9 respectively in the plurality of sub-regions r1-r9 (their symbols are omitted in FIG. 14) based on the data, as shown in FIG. 14. In the drawing, the density of dots (shading of dots) indicates the degree of transparency of the B-mode image. In particular, the degree of transparency of the B-mode image BI is lower for a higher density of dots (thicker dots) and higher for a lower density of dots (thinner dots).

The combined color elasticity images CEI1-CEI9 represent an exemplary embodiment of the image according to the angle in the present invention. They also represent an exemplary embodiment of the information indicating an angle between the direction of the acoustic line of ultrasound and direction of movement of the biological tissue in the present invention, and an exemplary embodiment of the information indicating a degree of match between the direction of the acoustic line of ultrasound and direction of movement of the biological tissue.

In the second embodiment, the image I including the combined color elasticity images CEI1-CEI9 may be a real-time image, or an image produced based on the B-mode image data (or B-mode data) and elasticity image data (or physical quantity data) stored the storage section 9.

According to the present embodiment, the operator may observe the combined color elasticity images CEI1-CEI9 to thereby recognize a displacement between the direction of the acoustic line of ultrasound and direction of movement of the biological tissue in each of the plurality of sub-regions r1-r9. In particular, the operator can recognize that the displacement between the direction of the acoustic line of ultrasound and direction of movement of the biological tissue is smaller for a lower degree of transparency of the B-mode image BI in the combined color elasticity images CEI1-CEI9. Therefore, the operator can understand which one(s) of the combined color elasticity images CEI1-CEI9 more accurately reflects elasticity of the biological tissue by the degree of transparency of the B-mode image BI. Thus, in case that the operator does not need to know local elasticity of a tumor or the like, such as a case in which he/she desires to know elasticity of the whole liver, he/she can find elasticity by referring to a combined color elasticity image in a sub-region having a higher degree of transparency of the B-mode image.

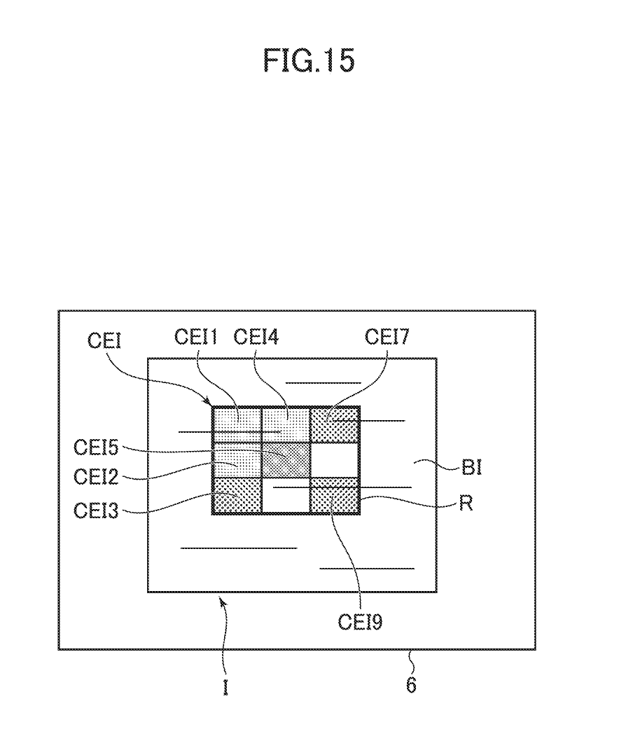

Next, a variation of the second embodiment will be described. The image display processing section 55 prevents display of the combined color elasticity images CEI1-CEI9 for those of the plurality of sub-regions r1-r9 having an angle θ1-θ9 of a prespecified angle θth or greater. In other words, the image display processing section 55 prevents display of the combined color elasticity images CEI1-CEI9 for those of the plurality of sub-regions r1-r9 not satisfying criteria that the angle θ1-θ9 should be smaller than the prespecified angle θth. For example, in case that the angles θ6, θ8 are equal to or greater than the prespecified angle θth, the image display processing section 55 does not display the combined color elasticity images CEI6, CEI8, as shown in FIG. 15.

The prespecified angle θth is set, for example, to an angle at which there is provided a combined color elasticity image inaccurately reflecting elasticity of the biological tissue and unnecessary for knowing its elasticity. The prespecified angle θth represents an exemplary embodiment of the prespecified threshold in the present invention. The criteria that the angle should be smaller than the prespecified angle θth represent an exemplary embodiment of the criteria regarding a prespecified threshold in the present invention.

Next, a third embodiment will be described. It should be noted that identical parts to those in the first or second embodiment will be omitted in the description.

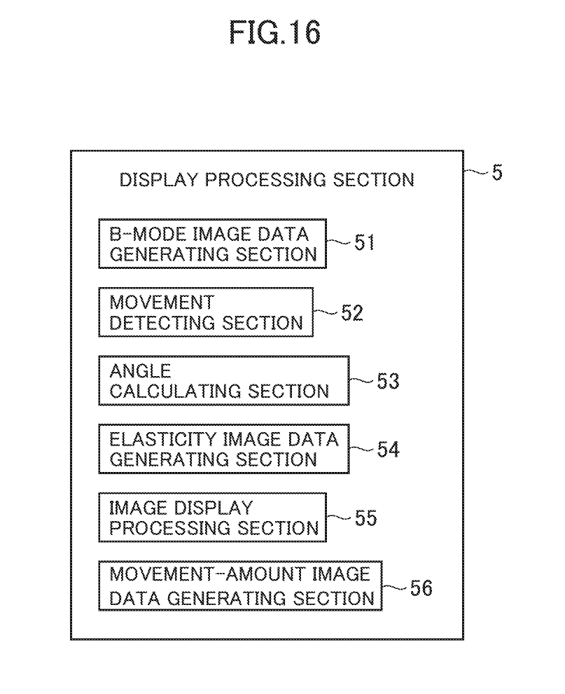

The display processing section 5 in the ultrasonic diagnostic apparatus in the present embodiment comprises a B-mode image data generating section 51, a movement detecting section 52, an angle calculating section 53, an elasticity image data generating section 54, an image display processing section 55, and in addition, a movement-amount image data generating section 56, as shown in FIG. 16. The movement-amount image data generating section 56 transforms data of the amount of movement of the biological tissue detected by the movement detecting section 52 into information representing colors, and applies scan conversion by the scan converter to generate movement-amount image data having information representing colors according to the amount of movement. The movement-amount image data generating section 56 gives multiple gradations to data of the amount of movement, and generates movement-amount image data comprised of information representing colors assigned to the gradations. The movement-amount image data generating section 56 represents an exemplary embodiment of the movement-amount image data generating section in the present invention.

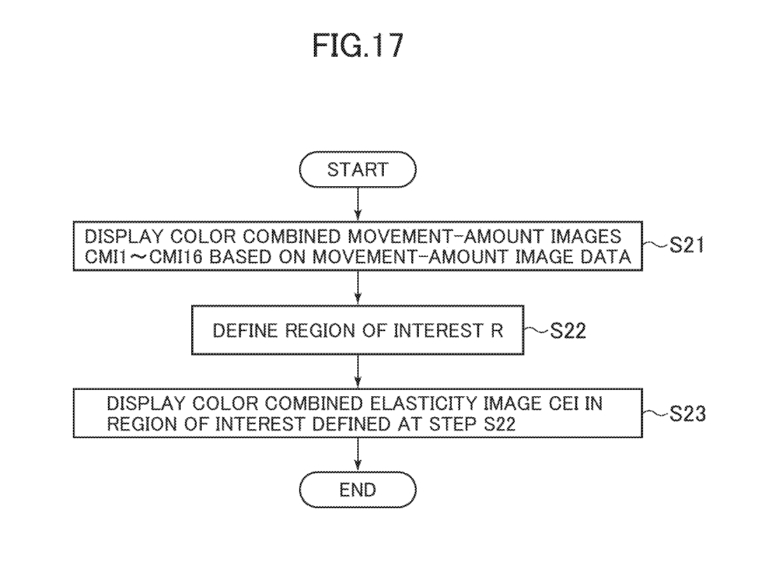

An operation of the present embodiment will now be described. In the present embodiment, after an image based on the movement-amount image data has been displayed, the position of a region of interest R in which an elasticity image is to be displayed is determined based on the image. Then, a combined color elasticity image CEI is displayed in the region of interest R. The operation will be particularly described with reference to the flow chart in FIG. 17.

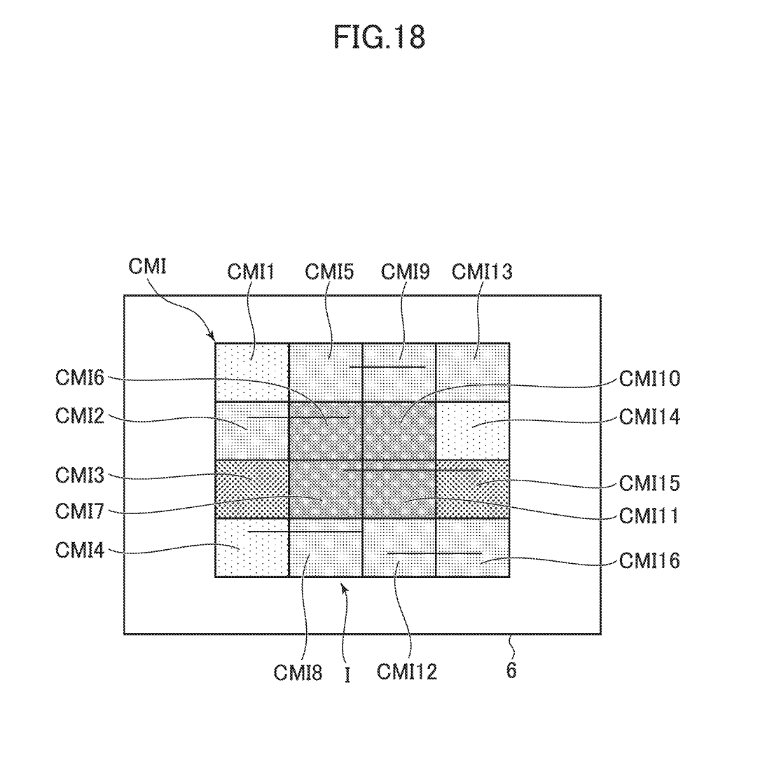

First, at Step S21, the display section 6 displays an image based on the movement-amount image data. The image is a combined color movement-amount image CMI of a combination of the movement-amount image data and B-mode image data. As shown in FIG. 18, the combined color movement-amount image CMI is comprised of combined color movement-amount images CMI1-CMI16 displayed respectively in a plurality of sub-regions r1-r16 (their symbols are omitted in FIG. 18) defined in a region displaying a B-mode image BI.

The display of the combined color movement-amount images CMI1-MI16 will be described in detail. First, transmission/reception of ultrasound by the ultrasonic probe 2 is conducted to generate B-mode image data. Similarly to the embodiments described earlier, the movement detecting section 52 calculates movement of the biological tissue in the B-mode image in each of the plurality of sub-regions r1-r16 based on B-mode image data in two temporally different frames to provide motion vectors v1-v16 (not shown).

Once the motion vectors v1-v16 have been obtained, the movement-amount image data generating section 56 generates movement-amount image data having a mode of display according to the amount of movement in the motion vectors v1-v16. Moreover, the angle calculating section 53 calculates angles θ1-θ16 between the direction of the acoustic line of ultrasound and the motion vectors v1-v16, respectively (−90≦θ1-θ16≦+90).

Next, the image display processing section 55 combines the movement-amount image data with the B-mode image data in a specified proportion to generate data of a combined color movement-amount image CMI. The image display processing section 55 generates data of the combined color movement-amount image CMI having respective degrees of transparency of the B-mode image BI according to the angles θ1-θ16 in the plurality of sub-regions r1-r16. Thus, combined color movement-amount images CMI1-CMI16 are produced respectively for the plurality of sub-regions r1-r16. Similarly to the embodiments described earlier, the combined color movement-amount images CMI1-CMI16 have a higher degree of transparency of the B-mode image BI for a greater absolute value of the angle θ1-θ16.

Once data for the combined color movement-amount images CMI1-CMI16 have been generated, the image display processing section 55 displays the combined color movement-amount images CMI1-CMI16 respectively in the plurality of sub-regions r1-r16 based on the data, as shown in FIG. 18. Again in the drawing, the shading of dots indicates the degree of transparency of the B-mode image BI. The combined color movement-amount images CMI1-CMI16 represent an exemplary embodiment of the image according to the angle in the present invention. The combined color movement-amount images CMI1-CMI16 represent an exemplary embodiment of the information indicating an angle between the direction of the acoustic line of ultrasound and direction of movement of the biological tissue in the present invention, and also an exemplary embodiment of the information indicating a degree of match of the direction of the acoustic line of ultrasound and direction of movement of the biological tissue.

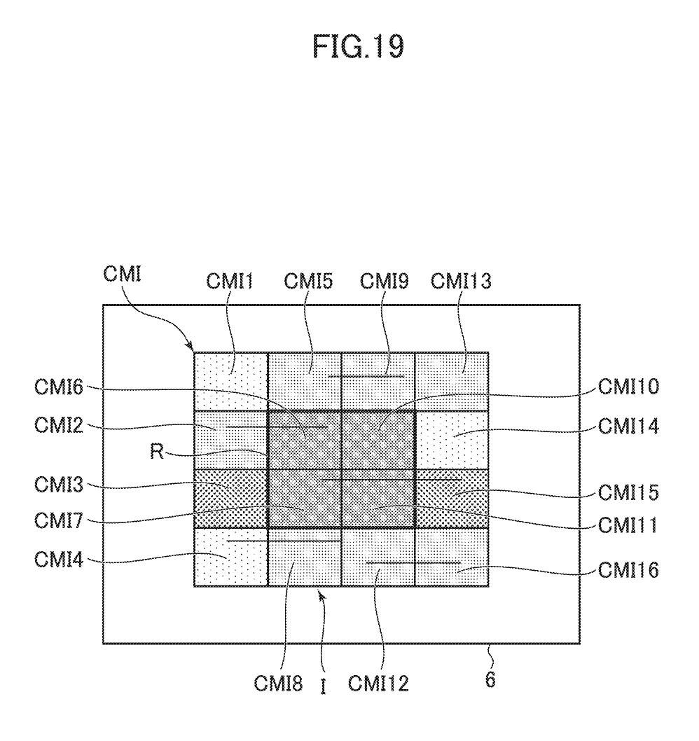

Next, at Step S22, the operator observes the combined color movement-amount images CMI1-CMI16 to define a region of interest R at a position for obtaining a combined color elasticity image CEI more accurately reflecting elasticity of the biological tissue. In particular, the operator defines a region of interest R in a sub-region having a lower degree of transparency of the B-mode image BI in the combined color movement-amount images CMI1-CMI16. For example, in case that the degree of transparency of the B-mode image BI in the combined color movement-amount images CMI6, CMI7, CMI10, CMI11 in the sub-regions r6, r7, r10, r11 is lower than those in other images, as shown in FIG. 19, the region of interest R is defined on the sub-regions r6, r7, r10, r11 in which the combined color movement-amount images CMI6, CMI7, CMI10, CMI11 are displayed.

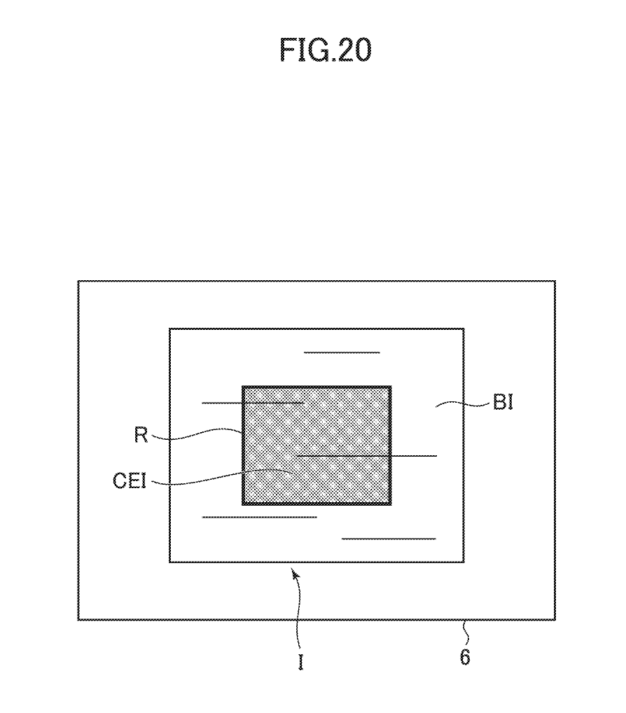

Once the region of interest R has been defined at Step S22 described above, transmission/reception of ultrasound for generating elasticity image data is conducted in addition to that for generating B-mode image data at Step S23. Then, the combined color elasticity image CEI is displayed in the region of interest R, as shown in FIG. 20.

According to the present embodiment, the operator may observe the combined color movement-amount images CMI1-CMI16 to thereby recognize a displacement between the direction of the acoustic line of ultrasound and direction of movement of the biological tissue in each of the plurality of sub-regions r1-r16. In particular, the operator can recognize that the displacement between the direction of the acoustic line of ultrasound and direction of movement of the biological tissue is smaller for a lower degree of transparency of the B-mode image BI in the combined color movement-amount images CMI1-CMI16. Therefore, the operator may define the region of interest R in a sub-region having a lower degree of transparency of the B-mode image BI to obtain an elasticity image more accurately reflecting elasticity of the biological tissue in the region of interest R.

While the present invention has been described with reference to the embodiments, it will be easily recognized that the present invention may be practiced with several modifications without departing from the spirit and scope thereof. For example, an arrow in a direction for the operator to move the ultrasonic probe and characters indicating the quantity (angle) of movement, etc., may be displayed in the display section 6 so that the direction of an acoustic line of ultrasound matches that of movement of the biological tissue.

1. An ultrasonic diagnostic apparatus, comprising:
an ultrasonic probe for conducting transmission/reception of ultrasound to/from biological tissue;
a strain calculating section for calculating a strain in several portions in said biological tissue based on two temporally different echo signals in an identical acoustic line acquired by said ultrasonic probe, said section calculating said strain in a direction of said acoustic line of ultrasound;
an elasticity image data generating section for generating data for an elasticity image according to the strain calculated by said strain calculating section;
a movement detecting section for detecting movement of said biological tissue in an ultrasonic image based on ultrasonic image data generated based on echo signals resulting from transmission/reception of ultrasound to/from said biological tissue;
an angle calculating section for calculating an angle between a direction of an acoustic line of ultrasound transmitted/received by said ultrasonic probe and a direction of movement of said biological tissue detected by said movement detecting section; and
a notifying section for notifying information based on the angle calculated by said angle calculating section.

2. The ultrasonic diagnostic apparatus as recited in claim 1, wherein said notifying section notifies information for allowing an operator to understand in which direction and at which angle to move said ultrasonic probe so that said direction of said acoustic line of ultrasound matches said direction of movement of said biological tissue.

3. The ultrasonic diagnostic apparatus as recited in claim 1, wherein said notifying section notifies information indicating an angle between said direction of said acoustic line of ultrasound and direction of movement of said biological tissue.

4. The ultrasonic diagnostic apparatus as recited in claim 1, wherein said notifying section notifies information indicating a degree of match between said direction of said acoustic line of ultrasound and direction of movement of said biological tissue.

5. The ultrasonic diagnostic apparatus as recited in claim 1, wherein
said movement detecting section detects said movement of said biological tissue in each of a plurality of sub-regions in said ultrasonic image, and said angle calculating section performs calculation of said angle in each of said plurality of sub-regions.

6. The ultrasonic diagnostic apparatus as recited in claim 5, wherein said notifying section displays an image according to said angle in a display section for each of said plurality of sub-regions.

7. The ultrasonic diagnostic apparatus as recited in claim 6, wherein said image according to said angle is an image produced using data of said elasticity image and having a mode of display according to said angle.

8. The ultrasonic diagnostic apparatus as recited in claim 6, wherein said notifying section prevents display of said image according to said angle in those of said plurality of sub-regions not satisfying criteria regarding a prespecified threshold defined for said angle.

9. The ultrasonic diagnostic apparatus as recited in claim 6, further comprising
a movement-amount image data generating section for generating data for a movement-amount image having a mode of display according to an amount of movement of said biological tissue detected at said movement detecting section, wherein
said image according to said angle is an image produced based on data of said movement-amount image and having a mode of display according to said angle.

10. The ultrasonic diagnostic apparatus as recited in claim 5, wherein said plurality of sub-regions are defined in a region of interest in which an image based on data of said elasticity image is displayed.

11. The ultrasonic diagnostic apparatus as recited in claim 5, wherein said plurality of sub-regions are defined in an ultrasonic image display region in which said ultrasonic image is displayed.

12. The ultrasonic diagnostic apparatus as recited claim 1, wherein said strain calculating section compares waveforms of two temporally different echo signals in an identical acoustic line acquired by said ultrasonic probe, and calculates a strain in several portions in said biological tissue based on a degree of distortion of the waveform associated with compression and relaxation of said biological tissue between said two echo signals.

13. The ultrasonic diagnostic apparatus as recited in claim 1, wherein said movement detecting section detects said movement of said biological tissue in said ultrasonic image based on a degree of similarity of ultrasonic image data between two different frames for an identical cross section generated based on echo signals obtained from transmission/reception of ultrasound to/from said biological tissue.

14. An ultrasonic diagnostic apparatus, comprising:
an ultrasonic probe for conducting transmission/reception of ultrasound to/from biological tissue; and
a processor configured to execute a plurality of functions, the functions comprising:
a strain calculating function configured to calculate a strain in several portions in said biological tissue based on two temporally different echo signals in an identical acoustic line acquired by said ultrasonic probe, said function further configured to calculate said strain in a direction of said acoustic line of ultrasound;
an elasticity image data generating function configured to generate data for an elasticity image according to the strain calculated by said strain calculating function;
a movement detecting function configured to detect movement of said biological tissue in an ultrasonic image based on ultrasonic image data generated based on echo signals resulting from transmission/reception of ultrasound to/from said biological tissue;
an angle calculating function configured to calculate an angle between a direction of an acoustic line of ultrasound transmitted/received by said ultrasonic probe and a direction of movement of said biological tissue detected by said movement detecting function; and
a notifying function configured to notify information based on the angle calculated by said angle calculating function.

15. A program for controlling an ultrasonic diagnostic apparatus including a processor, wherein said program is configured to cause the processor to execute:
a strain calculating function to calculate a strain in several portions in biological tissue based on two temporally different echo signals in an identical acoustic line acquired by an ultrasonic probe for conducting transmission/reception of ultrasound to/from said biological tissue, said function calculating said strain in a direction of said acoustic line of ultrasound;
an elasticity image data generating function to generate data for an elasticity image according to the strain calculated by said strain calculating function;
a movement detecting function to detect movement of said biological tissue in an ultrasonic image based on ultrasonic image data generated based on echo signals resulting from transmission/reception of ultrasound to/from said biological tissue;
an angle calculating function to calculate an angle between a direction of an acoustic line of ultrasound transmitted/received by said ultrasonic probe and a direction of movement of said biological tissue detected by said movement detecting function; and
a notifying function to notify information based on the angle calculated by said angle calculating function.
